(19) United States
(12) Patent Application Publication
(10) Pub. No.: US 2006/0084150 A1
(43) Pub. Date: Apr. 20, 2006

(54) Method for manufacturing maltose-rich products

(76) Inventor: Qunyu Gao, Guangzhou (CN)

Correspondence Address: Bingham McCutchen LLP, Suite 800, 1120 20th Street, NW, Washington, DC, 20036, US

(21) Appl. No.: 11/237,692

(22) Filed: Sep. 29, 2005

Related U.S. Application Data
(60) Provisional application No. 60/613,818, filed on Sep. 29, 2004.

(51) Int. Cl. C12P 19/02; C07C 31/18

(52) U.S. Cl. 435/105; 568/863

(57) ABSTRACT

The present invention provides a method for making highly pure maltose products from starch-containing raw materials (such as corn starch, potato starch, cassava starch, sweet potato starch, wheat starch, and rice starch etc.). The method includes a combined use of amylases and other malt hydrolyzing enzymes, a spray liquefaction method, an ion-exchange resin chromatography, a filtration, and a crystallization. The highly pure maltose products include maltose syrup, maltose alcohol, and solid maltose.

### RELATED APPLICATION

The present application claims the priority of U.S. Provisional Application No. 60/613,818, filed on Sep. 29, 2005, which is herein incorporated by reference.

### FIELD OF THE INVENTION

The present relates to a method for manufacturing starch and starch sugars, in particular, it pertains to a method for manufacturing maltose-rich products, including, but not limited to, maltose-rich syrup, maltose alcohol and solid maltose.

### BACKGROUND OF THE INVENTION

Maltose is a valuable raw material in the production of maltitol (α(1→4) glucosyl sorbitol), which is a sugar alcohol generally used as a sweetening agent in low-caloric, dietary and low-cariogenic foods, such as confectionary products and chewing gums. Maltitol is prepared in the form of crystalline maltitol or maltitol syrup.

A maltose-rich syrup has a wide range of application as a nutritious sweetener and filler in the food industry, being used for example in gourmet candies, health drinks, solutions for oral administration, milk and malt extracts, deserts, beverages and other areas. Due to the fact that maltose is less likely to produce acid from the decomposition of decayed teeth caused by bacteria, it can be used to prevent cavities. In the pharmaceutical industry, it can be used to prepare various types of Chinese medicine and cough syrups having the effects of soothing the lungs, alleviating stomach pain and coughing, etc.

Conventionally, maltose, has been available as a saccharified starch product with a maltose content of about 40-50 w/w % based upon the weight of the dry solid solute, which is obtainable by subjecting a liquefied starch solution to the action of a malt enzyme.

A maltose-rich syrup can be used to manufacture maltose alcohol following a hydrogenation treatment and it can also be used to manufacture pure maltose. Maltose-rich alcohol has an anti-tooth decay property and can be used as a low-calorie functional sweetener and a fat substitute in the production of gourmet anti-cavity hard candies, toffee, gummy candies and sugar-free foodstuffs, etc. As a raw material for chemical synthesis, it has already been used for the production of synthesized resin surfactants and contact agents, etc. In cosmetics, it can be used as a regulator for the degree of moisture. Due to the fact that the degree of increase in the blood sugar and the insulin level in the blood is very small on average following the intake of maltose alcohol by the human body and that it can promote the absorption of calcium, it can be developed for use in food that is designed specifically for patients who suffer from diabetes, hepatic disease*, cardiovascular disease, arteriosclerosis, high blood pressure, obesity and osteoporosis. In addition, maltose alcohol is often used as a flavoring agent in tablets that are rapidly dissolvable in the mouth.

A maltose-rich syrup is a new nutritious sugar product with a wide range of applications. It is characterized by being mildly sweet, having a unique flavor and it can be easily digested and absorbed. Its main component is maltose and at the same time, it contains a small amount of glucose and oligosaccharide. Depending on the content of maltose and the degree of refinement, it can be roughly divided into maltose (with a content of maltose of 40-50%), high maltose (50-70%), ultrahigh maltose (more than 70%) and crystal maltose, etc.

The conventional method of manufacturing maltose syrup is hydrolysis of starch with enzymes, and the main process of the technology is as follows: starch slurry→liquefaction→cooling→saccharification→heating→filtration→ion exchange→concentration under vacuum→product.

At the present time, for maltose syrup manufactured on an industrial scale, the content of the maltose is generally in the range of 50-70%. Nickel catalyst is added under alkaline condition, at 5-10% of the amount of starch added to a colorless pure high maltose syrup with a solids content of 40-60%. Hydrogen gas is blown into a high-pressure reactor at 5-12 Mpa, the maltose starts to absorb the hydrogen gas to carry out a hydrogenation reaction and a maltose alcohol can be obtained. Next, the catalyst in the syrup is filtered and removed, and following a treatment with activated carbon and ion exchange, a clear maltose alcohol can be obtained. Finally, processes including concentration under vacuum, crystallization, separation, and drying are carried out, and a maltose alcohol in liquid and powder forms is produced.

In the preparation of the maltose syrup, the following disadvantages have been observed: (1) the DE value is too high after the starch is liquefied; (2) the presence of glucose in the syrup affects the quality of the product, due to its hindering of the formation of crystals; and (3) the viscosity of the syrup is reduced. Also, during the process of saccharification, if hydrolysis with enzymes is not carried out thoroughly, the content of the oligosaccharide with DP≧3 will be too high. At the same time, the maltose syrup in its viscous state causes great inconvenience in the storage and transportation of the product.

The presently known technology for making highly (≧98%) maltose-rich requires the use of a maltose syrup with at least 85% maltose content. The maltose syrup would then go through an adsorption, an ion exchange chromatography, a membrane separation, and finally a crystallization process. The technical process is complicated. The cost of production is very high and the range of application is limited (for use mainly in medical treatment in injections for patients suffering from diabetes and high blood pressure).

The present invention provides a novel process for producing maltose-rich using normal starch (such as corn starch, potato starch, cassava starch, sweet potato starch, wheat starch and rice starch etc.) as starting material. The process uses a combination of enzymes, coupled with high-temperature spray technology, differential liquefaction and saccharification, to obtain maltose-rich syrup with a maltose content of 90% or higher. The maltose product is of high quality, the yield is effectively increased and the cost of production is significantly reduced. The present invention uses a simple technical process with a low cost of production. The final product is a solid, and it is easy to store and transport, thus providing a wider range of application compared with the maltose syrup.

### SUMMARY OF THE INVENTION

The objective of the present invention is to overcome the disadvantages of the prior art by providing a method of manufacturing maltose-rich products. The maltose-rich products as described in the present invention include maltose-rich syrup, maltose alcohol and solid maltose.

In the present invention normal starches (such as corn starch, potato starch, cassava starch, sweet potato starch, wheat starch and rice starch, etc.) are used as the raw material, with a combined use of multiple enzymes, coupled with a high-temperature spraying technology, differential liquefaction and saccharification to obtain a maltose-rich syrup with a maltose content of 90% or more.

The present invention provides a method for making a maltose syrup comprising the steps of (1) dispersing a starch-containing raw material in a solution to form a starch slurry; (2) adding a first α-amylase to the starch slurry to form a reaction mixture; (3) spray liquefying the reaction mixture to form a liquefied reaction mixture; (4) adding a malt hydrolyzing enzyme group to the liquefied reaction mixture to form a hydrolyzed reaction mixture; and (5) adding a second α-amylase to the hydrolyzed reaction mixture to form the maltose syrup.

The starch-containing raw material is at least one of corn starch, potato starch, cassava starch, sweet potato starch, wheat starch, and rice starch.

The first α-amylase is a high temperature α-amylase.

The malt hydrolyzing enzyme group comprises β-amylase, pullanase, and malt trisaccharide hydrolysis enzyme.

The second α-amylase is a medium temperature α-amylase.

The present invention further provides a method for making a maltose alcohol which is made by adding a catalyst to the maltose syrup to form a catalyzed maltose syrup, and then infusing a hydrogen gas to the catalyzed maltose syrup to form the maltose alcohol.

The preferred catalyst includes a nickel catalyst or a nickel aluminum molybdenum catalyst complex.

The present invention also provides a method for making a solid maltose by passing the maltose alcohol through an ion-exchange resin chromatography to form a purified maltose alcohol; passing said purified maltose alcohol through a filter to collect a filtrate; concentrating the filtrate to collect a concentrate; crystallizing the concentrate to form a solid maltose.

The maltose products produced according to the present method are of high quality. The yield is effectively increased and the cost of production is significantly reduced.

### DETAILED DESCRIPTION OF THE INVENTION

The present invention uses raw material that can be found anywhere in the market, including, but not limited to any kind of starch, such as corn starch, potato starch, cassava starch, sweet potato starch, wheat starch and rice starch, etc. The method is described in general as follows: The raw material is dispersed in water, the solids content of the dispersed raw material is about 15-40%. The dispersed raw material is adjusted to adequate pH and temperature. A high-temperature α-amylase is added to the dispersed raw material. The mixture of the raw material and the high-temperature α-amylase is spray liquefied. During the saccharification process, β-amylase, malt trisaccharide enzyme, pullanase and medium-temperature amylase, etc., are added in separate steps to carry out a concerted catalysis.

In order to ensure a proper DE value after the liquefaction process, the high-temperature α-amylase must be added at two separate occasions. The DE value can be determined by controlling the iodine number in the liquefaction. The liquefaction process destroys the molecular bond of the starch in the native starch granules, paving the way for saccharification so that more maltose can be produced. After the liquefaction process, β-amylase, malt trisaccharide enzyme, pullanase and medium-temperature amylase are added to carry out a concerted saccharification reaction in separate steps, the operation temperature, pH and reaction time must be adjusted so that optimal conditions are ensured for the various enzyme activities.

All of these parameters are determined based on the sources of the substrate used, the source, type and activity of the enzymes, the concentration of the substrate and on whether or not a suppressing agent is added. It is necessary to denature the enzymes after the saccharification reaction, [the temperature can be adjusted to]≧75° C. for 15 min or the pH can be adjusted to ≦3.0 for about 1 h. The impurities and by-products of the reaction are removed by filtration, ion exchange and other processes and, following further concentration, a maltose-rich syrup is obtained; if a certain amount of seed crystal is added, by means of a crystallization process, a solid maltose crystal product can be obtained; and by means of hydrogenation, refining, concentration, crystallization, separation, drying and other processes, a corresponding maltose alcohol can be obtained.

The following method for manufacturing a maltose-rich product is illustrative, but not limiting the scope of the present invention. Reasonable variations, such as those occur to reasonable artisan, can be made herein without departing from the scope of the present invention.

(1) A high temperature α-amylase at an amount of 2.5-10.5 LU/g starch was added to a starch slurry having a concentration of 15-40%, pH 5.2-6.2, to form a starch slurry mix. The starch slurry and the high temperature α-amylase were stirred for about 15-30 min.

(2) The starch slurry mix was spray liquefied twice, with the first time at about 115° C., for about 30 second; and the second time at 125° C., for 10 min.

(3) The spray liquefied starch slurry mix was cool to 85-95° C. Additional high-temperature α-amylase was added, followed by a third time spray liquefaction at about 135° C., for about 5 min.

(4) The spray liquefied starch slurry mix was cool to 58-62° C. The pH was adjusted. β-amylase, pullanase and malt trisaccharide hydrolysis enzyme were added to the spray liquefied starch slurry mix. The hydrolysis was carry out hydrolysis for 24-48 h. The amount of the enzymes used are 0.3-3 DP/g starch, 0.08-0.8 PUN/g starch and 0.8-8.0 MANU/g starch, respectively:

(5) A medium-temperature amylase is added, 0.028-0.28 KNU/g starch, and saccharification for up to 60 hours. At this point, a colorless, maltose-rich syrup with a 90-96% maltose content in the syrup is obtained. This maltose syrup contained about 40-60% solids.

(6) A 5 to 10% of Nickel catalyst was added to the maltose-rich syrup, under alkaline condition, in a high-pressure reactor.

(7) Hydrogen gas was infused into the high-pressure reactor at 5-12 Mpa. The maltose started to absorb the hydrogen gas and carried out a hydrogenation reaction. A maltose alcohol was generated.

(8) The nickel catalyst in the syrup was filtered and removed. After a treatment with activated carbon and ion exchange, a clear maltose alcohol was obtained.

(9) The maltose alcohol was concentrated under vacuum, crystallized, separated, and dried. A maltose-rich alcohol in liquid or powder forms was obtained.

The above mentioned technologies, including spray liquefaction, hydrogenation, filtration, refinement, concentration, crystallization, separation and drying are all standard technologies known to one of ordinary skill in the art.

A solid maltose product with high purity was obtained by heating the maltose syrup (containing greater than 70% maltose) to 70-80° C., followed by adding activated carbon at the amount of 0.1-1.0% of the weight of the solid to the maltose syrup and filtered. The filtrate was evaporated to about 75-85% of the weight of the dry solid. A seed crystal with a granule size of about 120-150 mesh at the amount of 0.05-0.5% by weight of the dry solid in the maltose syrup was added to the filtrate and stirred thoroughly; the filtrate was then poured into a mold, cooled naturally to the ambient temperature and a solid maltose was formed.

The obtained solid maltose crystal product has a stable structure and shape, is easily dissolved in water and returns to a maltose syrup after being dissolved. It has the same functions as maltose syrup and is adapted for use in all the areas where a maltose syrup can be used, in particular as a forming agent for medicine and an additive in food.

Compared to the state of the art, the present invention has the following significant advantages:

1. By means of the triple spray liquefaction, multiple enzyme cooperation, stepwise liquefaction and saccharification, a maltose-rich syrup with a >90% maltose content and its corresponding alditol can be obtained using a simple technical process and having a low cost of production;

2. The maltose syrup can be used directly as the raw material to produce a solid maltose crystal product which prevents a complicated refinement process by providing a simple preparation method and low cost of production;

3. The solid maltose produced by the present invention has a wider range of application compared to the maltose syrup of the state of the art and at the same time it is easy to store and transport.

The present invention is further explained below with reference to the application examples:

### APPLICATION EXAMPLE 1

(1) High-temperature α-amylase at an amount of 6.5 LU/g starch was added to a starch slurry to form a reaction sample with a concentration of 25%, pH 5.8 and stirred for about 20 min;

(2) The spray liquefaction was carried out twice, the first time at 115° C. for 30 seconds; the second time at 125° C. for 10 min;

(3) The reaction sample was cooled to 92° C. Additional high-temperature α-amylase was added, followed by spray liquefaction for the third time at 135° C. for 5 min;

(4) The reaction sample was cooled to 58° C. and the pH was adjusted. P-amylase in the amount of 1.5 DP/g starch, pullanase in the amount of 0.6 PUN/g starch, and malt trisaccharide hydrolysis enzyme in the amount of 4.0 MANU/g starch were added to carry out hydrolysis for 24-48 hours;

(5) Medium temperature α-amylase in the amount of 0.2 KNU/g starch was added to the reaction sample, and saccharification was carried out for up to 60 h;

(6) At this point, a maltose-rich syrup with a 94.5% maltose content in the syrup was obtained.

(7) The above maltose syrup was subjected to a decoloration treatment with activated carbon (75-80° C., 30 min);

(8) An ion-exchange resin treatment and a filtration with a 0.45-μm membrane was carried out;

(9) The filtrate was concentrated to 78-85% to obtain a maltose-rich syrup with a 94.5% maltose content.

### APPLICATION EXAMPLE 2

(1) A high-temperature α-amylase at an amount of 4.5 LU/g starch was added to a potato starch slurry with a concentration of 15% and pH 5.4 and stirred for 15 min to form a reaction sample;

(2) The spray liquefaction was carried out twice, the first time 115° C., 30 s; the second time 125° C., for 10 min;

(3) The reaction sample was cooled down to 90° C., and additional high-temperature α-amylase was added, followed by another spray liquefaction at 135° C. for 5 min;

(3) The reaction sample was then cooled to 60° C. with the pH adjusted; β-amylase in the amount of 1.2 DP/g starch, pullanase in the amount of 0.5 PUN/g starch, and malt trisaccharide hydrolysis enzyme in the amount of 3.5 MANU/g starch were added to carry out hydrolysis for 24-48 hours;

(4) A medium-temperature α-amylase in the amount of 0.18 KNU/g starch was added, and saccharification is carried out for up to 60 hours;

(5) At this point, a maltose-rich syrup with a 96% maltose content in the syrup was obtained.

(6) The pH of the reaction sample was adjusted and a nickel aluminum molybdenum catalyst complex is added to carry out a reduction reaction at a prescribed temperature and under hydrogen gas pressure to obtain a corresponding maltose alcohol;

(7) The maltose alcohol solution was subjected to a decoloration treatment with activated carbon (75-80° C., 30 min);

(8) The maltose alcohol solution was undergone an ion-exchange resin treatment and a fine filtration with a 0.45-μm membrane;

(9) The filtrate was concentrated to 78-85%;

(10) The concentrated filtrate was cooled down to 50-55° C. and then continuous crystallization was carried out in a horizontal crystallization tank;

(11) The concentrated and crystallized sample was separated and dried to obtain a maltose alcohol in powder form.

### APPLICATION EXAMPLE 3

(1) A high-temperature α-amylase at an amount of 7.2 LU/g starch was added to a cassava starch slurry with a concentration of 30%, pH 5.6 and stirred for 25 min to obtain a reaction sample;

(2) The reaction sample was spray liquefaction twice, the first time at 115° C. for 30 seconds; the second time at 125° C. for 10 min;

(3) The reaction sample was cooled to 88° C.; additional high-temperature α-amylase was added, followed by another spray liquefaction at 135° C. for 5 min;

(4) The reaction sample was cooled to 62° C. and the pH was adjusted. β-amylase in the amount of 2.0 DP/g starch, pullanase in the amount of 0.7 PUN/g starch; and malt trisaccharide hydrolysis enzyme in the amount of 5.5 MANU/g starch were added to carry out hydrolysis for 24-48 hours;

(5) A medium-temperature α-amylase in the amount of 0.20 KNU/g starch was added, and saccharification was carried out for up to 60 hours;

(6) At this point, a maltose-rich syrup with a 92.5% maltose content in the syrup [is obtained].

(7) The pH of the reaction sample was adjusted and a nickel aluminum molybdenum catalyst complex was added to carry out a reduction reaction at a prescribed temperature and under hydrogen gas pressure to obtain a corresponding maltose alcohol;

(8) The maltose alcohol was subjected to a decoloration treatment with activated carbon (75-80° C., 30 min);

(9) The decolored maltose alcohol was further purified by an ion-exchange resin chromatography and a fine filtration with a 0.45-μm membrane;

(10) The filtrate was further concentrated until the concentration was 78-85%;

(11) The temperature of the concentrate was lower to 50-55° C. and then continuous crystallization was carried out in a horizontal crystallization tank;

(12) The crystallized maltose alcohol was separated and dried to obtain a maltose alcohol product in powder form.

### APPLICATION EXAMPLE 4

(1) A high-temperature α-amylase at an amount of 10 LU/g starch was added to a sweet potato, wheat and rice starch slurry with a concentration of 40% and pH 5.7 and stirred for 30 min to form a reaction sample;

(2) The reaction sample was spray liquefaction twice, the first time at 115° C. for 30 seconds; the second time at 125° C. for 10 min;

(3) The reaction sample was cooled to 95° C.; additional high-temperature α-amylase was added, followed by spray liquefaction for the third time at 135° C. for 10 min;

(4) The reaction sample was cooled to 60° C., and the pH was adjusted; P-amylase at the amount of 3.0 DP/g starch, pullanase at the amount of 0.5 PUN/g starch, and malt trisaccharide hydrolysis enzyme at the amount of 7.5 MANU/g starch were added to carry out hydrolysis for 24-48 hours;

(5) A medium-temperature α-amylase at the amount of 0.26 KNU/g starch was added, and saccharification was carried out for up to 60 h;

(6) At this point, a maltose-rich syrup with a 90.5% maltose content in the maltose syrup was obtained;

(7) The pH of the maltose syrup was adjusted and a nickel aluminum molybdenum catalyst complex was added to carry out a reduction reaction at a prescribed temperature and under hydrogen gas pressure to obtain a corresponding maltose alcohol;

(8) The maltose alcohol was subjected to a decoloration treatment with activated carbon (75-80° C., 30 min);

(9) The decolored maltose alcohol was further purified by an ion-exchange resin chromatography and a fine filtration with a 0.45-μm membrane;

(10) The filtrate was further concentrated until the concentration was 78-85%;

(11) The temperature of the concentrate was lower to 50-55° C. and then continuous crystallization was carried out in a horizontal crystallization tank;

(12) The crystallized maltose alcohol was separated and dried to obtain a maltose alcohol product in powder form.

### APPLICATION EXAMPLE 5

The maltose syrup with a maltose content of about 76% that was obtained in Application Example 1, in which the content of glucose was 11.3% and the content of the tri- or higher saccharide was 13.7%, was heated at a certain temperature; 0.5% activated carbon was added and stirred for 30 min, and filtered; The filtrate was evaporated until a maltose solution was obtained in which the dry solids content was 78.5%; the temperature was then reduced to 65° C.; a seed crystal was added with a granule size of about 120-150 mesh and the amount used was 0.1% of the dry solid in the solution; the solution was stirred thoroughly to mix the seed crystal; the solution was then poured into a mold, cooled naturally to the ambient temperature and a solid maltose was formed.

### APPLICATION EXAMPLE 6

The maltose syrup with a maltose content of about 83% that was obtained in Application Example 2, in which the content of glucose was 8.9% and the content of the tri- or higher saccharide was 8.1%, was heated at a certain temperature, 0.9% activated carbon was added, stirred for 30 min, and filtered. The filtrate was evaporated until a maltose solution was obtained in which the dry solids content was 82.5%; the temperature was then reduced to 60° C. A seed crystal was added with a granule size of about 120-150 mesh and the amount used was 0.3% of the dry solid in the solution; the solution was stirred thoroughly to mix the seed crystal; the syrup was then poured into a mold, cooled naturally to the ambient temperature and a solid maltose was formed.

While the invention has been described by way of examples and in terms of the preferred embodiments, it is to be understood that the invention is not limited to the disclosed embodiments. On the contrary, it is intended to cover various modifications as would be apparent to those skilled in the art. Therefore, the scope of the appended claims should be accorded the broadest interpretation so as to encompass all such modifications.

We claim:

1. A method for making a maltose syrup comprising:
dispersing a starch-containing raw material in a solution to form a starch slurry;
adding a first α-amylase to said starch slurry to form a reaction mixture;
spray liquefying said reaction mixture to form a liquefied reaction mixture;
adding a malt hydrolyzing enzyme group to said liquefied reaction mixture to form a hydrolyzed reaction mixture; and
adding a second α-amylase to said hydrolyzed reaction mixture to form said maltose syrup.

2. The method according to claim 1, wherein said starch-containing raw material is at least one selected from the group consisting of corn starch, potato starch, cassava starch, sweet potato starch, wheat starch, and rice starch.

3. The method according to claim 1, wherein said first α-amylase is a high temperature α-amylase.

4. The method according to claim 1, wherein said malt a malt hydrolyzing enzyme group comprises β-amylase, pullanase, and malt trisaccharide hydrolysis enzyme.

5. The method according to claim 1, wherein said second α-amylase is a medium temperature α-amylase.

6. A method for making a maltose alcohol comprising:
adding a catalyst to said maltose syrup according to claim 1 to form a catalyzed maltose syrup,
infusing a hydrogen gas to said catalyzed maltose syrup to form said maltose alcohol.

7. The method according to claim 6, wherein said catalyst is a nickel catalyst or a nickel aluminum molybdenum catalyst complex.

8. A method for making a solid maltose comprising:
passing said maltose alcohol according to claim 6 through an ion-exchange resin chromatography to form a purified maltose alcohol;
passing said purified maltose alcohol through a filter to collect a filtrate;
concentrating said filtrate to collect a concentrate;
crystallizing said concentrate to form a solid maltose.
